SEQUENCE LISTING (8 sequences)

SEQ ID NO: 1 - DNA, 978 bp, VP7 gene of human rotavirus
  CDS (1)..(978)
atgtatggtattgaatataccacaattctaacttttttgatatcacttgtatttattaattatatagtgaaatcaataactagaacaatggactttgttatctatagatttttgtttgttatagttgtactagcaccattcattaaaacgcaaaactatggaataaacttgccgataactggttctatggatacaccatatatgaattcaacaatgagtgaaacattcttaacttctactttatgtttatattacccgaacgaagcagcaactcagatcgcagacgacaagtggaaagatactctctcacaactttttctcactaaagggtggccaacaggatcggtttactttaaagaatatacagatgttgcatcattttccgtagatccacaactatattgtgattataatattgtaatgatgaaatatgatggaaattcacaactagatatgtctgaattggctgatttaatattaaatgaatggctatgtaatccaatggatatagctctttactattatcaacaaacagatgaagcaaataaatggatatcaatgggcgattcatgtacgataaaagtatgcccactcaatacacagacccttggaataggatgttcgactactgatccaacaacatttgaggaagtggcttctgcagagaaattggtgataacagatgttgtagatggagttaaccacaaactagatgtgacaaccgctacgtgtacaataagaaactgtaaaaagcttggaccaagagaaaatgttgcgataattcaagtaggaggttcaaatatactcgatattacagcagatccaactacagctccacaaactgaaagaatgatgcgtataaattggaaaaaatggtggcaagtgttttatactatagtcgattatgttaatcaaattgtacaagtgatgtccaaacgatcacgttctttaaattccgctgctttttattatcgagtc

SEQ ID NO: 2 - protein, 326 aa, VP7 gene of human rotavirus
MYGIEYTTILTFLISLVFINYIVKSITRTMDFVIYRFLFVIVVLAPFIKTQNYGINLPITGSMDTPYMNSTMSETFLTSTLCLYYPNEAATQIADDKWKDTLSQLFLTKGWPTGSVYFKEYTDVASFSVDPQLYCDYNIVMMKYDGNSQLDMSELADLILNEWLCNPMDIALYYYQQTDEANKWISMGDSCTIKVCPLNTQTLGIGCSTTDPTTFEEVASAEKLVITDVVDGVNHKLDVTTATCTIRNCKKLGPRENVAIIQVGGSNILDITADPTTAPQTERMMRINWKKWWQVFYTIVDYVNQIVQVMSKRSRSLNSAAFYYRV

SEQ ID NO: 3 - DNA, 434 bp, VP4 gene of human rotavirus
  CDS (1)..(429)
tatgacgaaatagaacagattggatcggagaaaactcaaaatgtaacgataaatccaggtccatttgcacagactagatatgctccagttgattggggacacggagagattaatgattcaactacagtggaaccagttttagatggtccttatcaacccactgcattcaaaccacccaatgattattggctgcttattagctcaaatacggatggagtggtctatgaaagtacaaataatagtgacttttggacagcagttatcgctgtcgaaccacatgtcagtcaaacaaataggcaatatgttttatttggtgagaacaagcagtttaatgtagaaaataattcagataaatggaaatttttcgaaatgtttaaaggtagtagtcagagtgatttttctaatagacggactctaacctctaaacaatagaa

SEQ ID NO: 4 - protein, 143 aa, VP4 gene of human rotavirus
YDEIEQIGSEKTQNVTINPGPFAQTRYAPVDWGHGEINDSTTVEPVLDGPYQPTAFKPPNDYWLLISSNTDGVVYESTNNSDFWTAVIAVEPHVSQTNRQYVLFGENKQFNVENNSDKWKFFEMFKGSSQSDFSNRRTLTSKQ

SEQ ID NO: 5 - DNA, 28 bp, Artificial Sequence
  Sense primer for VP7
ggctttaaaagagagaatttccgtctgg

SEQ ID NO: 6 - DNA, 27 bp, Artificial Sequence
  Anti-sense primer for VP7 gene
ggtcacatcatacaattctaatctaag

SEQ ID NO: 7 - DNA, 22 bp, Artificial Sequence
  Sense-primer for VP4 gene
tggcttcgccattttatagaca

SEQ ID NO: 8 - DNA, 20 bp, Artificial Sequence
  Anti-sense primer for VP4 gene
atttcggaccatttataacc

(19) United States
(12) Patent Application Publication
(10) Pub. No.: US 2008/0293039 A1
(43) Pub. Date: Nov. 27, 2008

(54) VP7 gene of human rotavirus and composition for diagnosis of human rotavirus infection comprising primer or probe specific to thereof

(75) Inventors: Soon-Young Paik, Seoul (KR); Jin Han Kang, Seoul (KR)

Correspondence Address: BUCHANAN, INGERSOLL & ROONEY PC, POST OFFICE BOX 1404, ALEXANDRIA, VA, 22313-1404, US

(73) Assignee: MSD Korea Co., Ltd., Seoul (KR)

(21) Appl. No.: 12/000,029

(22) Filed: Dec. 7, 2007

(30) Foreign Application Priority Data
Mar. 29, 2007 (KR) 10-2007-0031079

(51) Int. Cl. C12Q 1/70; C07H 21/04

(52) U.S. Cl. 435/5; 536/23.72

(57) ABSTRACT

The present invention relates to a VP7 gene of human rotavirus and a composition for diagnosis of human rotavirus infection comprising primer or probe specific to thereof, and more particularly to a VP7 gene encoding the amino acid sequence set forth in SEQ ID NO:2 and a composition for diagnosis of human rotavirus infection comprising primer or probe specific to thereof. The human rotavirus VP7 gene according to the present invention will be useful for diagnosis of novel G11 type human rotavirus infection, and will be used for development of rotavirus vaccine.

### TECHNICAL FIELD

The present invention relates to a VP7 gene of human rotavirus and a composition for diagnosis of human rotavirus infection comprising primer or probe specific to thereof, and more particularly to a VP7 gene encoding the amino acid sequence set forth in SEQ ID NO:2 and a composition for diagnosis of human rotavirus infection comprising primer or probe specific to thereof.

### BACKGROUND ART

Rotavirus is the most common cause of severe diarrhoeal disease in infants and young children all over the world. About 40% of more than 125 million cases of diarrhoea each year in the world are attributed to rotavirus (World Health Organization: WHO WER 74:33-38, 1999). According to reports of the Center for Disease Control and Prevention (CDC) in the United States, each year, rotavirus causes approximately 111 million infections, 25 million hospitalizations, and 440,000 deaths in children <5 years of age, worldwide, and the total associated medical costs due to rotavirus infection are estimated at US $10 millions in the United States (Bresee J et al., Emerg Infect Dis 10:988-995, 2004). Therefore, the World Health Organization(WHO) takes a first project for producing a rotavirus vaccine to reduce rotavirus infection in developing countries and to curtail medical costs in developed countries (Glass RI et al., Science 265:1389-1391, 1994).

In Korea, rotavirus is also the most common cause of severe diarrhoeal disease in infants and young children. In order to develop the vaccine for rotavirus, it is earnestly necessary to obtain information on clinical characteristics by rotavirus infection and distribution of recently widespread rotavirus genotypes in Korean infants and young children. In order to determine distribution of rotavirus genotypes in Korea, rotavirus-infected samples were collected and analyzed from July 2002 to June 2003. As a result, rotavirus a genotype G4P6 was the most frequently distributed (22%) in Korea while the genotype is rarely found in the world, and a new rotavirus genotype G9P8 (11%) was found. Therefore, on the basis that the rotavirus infection in Korea is caused by the new rotavirus genotype or the globally rare rotavirus genotype, there has been a need for the development of a rotavirus vaccine adapted for Korea.

Rotavirus, a member of the family Reoviridae is a non-enveloped, icosahedral, and composed of a core, a middle capsid and an outer capside. The core contains VP1, VP2 and VP3 proteins encoded by RNA segments 1, 2 and 3, respectively. The middle capsid contains a VP6 protein encoded by a RNA segment 6. The outer capside contains a VP7 protein encoded by a RNA segment 9 (in certain circumstances, RNA segment 7 or 8 depending on the stain), and a VP4 spike protein encoded by a RNA segment 4.

Rotavirus is classified into 7 serotypes from group A to group G depending on antigenicity of VP6 protein, and the most common group in the worldwide, group A is reclassified into G type (glycoprotein type) by VP7 protein and P type (protease-sensitive type) by VP4 protein. The protein making up the middle capsid, VP6 is a main protein of rotavirus and targeted by antigen diagnosis analysis. Currently, 15 kinds of G serotypes and 24 kinds of P genotypes have been identified in human, mammals and fowls, and the most common rotavirus in human is a human rotavirus combining G1, G2, G3, G4 and G9 in G types, with P[8] and P[4] in P types.

Since a G11 type rotavirus (YM strain) was firstly isolated from pigs in Mexico in 1983 (Ruiz et al., Journal of Virology, 62(11) p4331-4336, 1988), it was subsequently identified from human, combined with P[25] in Bangladesh (Rahman et al., Journal of Clinical Microbiology, 43(7), p3208-3212, 2005). In Korea, there is no isolated case of G11 type human rotavirus, and G11 P[4] type human rotavirus has not been detected yet.

Meanwhile, it is difficult to develop vaccines for protecting from infections of all serotypes of rotavirus because cross-protection between serotypes of rotavirus is failed (Glass RI et al., J Infect Dis 192 Suppl 1:S160-166, 2005). Attenuated oral live vaccines (cow, UK, WC3; monkey, SA 11, MMU18006; human, M37) and animal-human recombinant vaccines which have been developed until now do not show sufficient protection ability against infections of other serotypes yet (Anderson EL et al., J Infect Dis 153:823-831, 1986; Bernstein DI et al., JAMA 273:1191-1196, 1995; Clark HF et al., Am J Dis Child 140:350-356, 1986; Conner ME et al., Curr Top Microbial Immunol 105:253, 1994; De Mol P. et al., Lancet II 108, 1986; Flores J. et al., J Clin Microbiol 27: 512-518, 1988; Kapikian AZ et al., Adv Exp Med Biol 257:67, 1990; Rennels MB et al., Pediatrics 97:7-13, 1996; Vesikari T, Vaccine 11:255-261, 1993). Recently, Rotashiled® (Wyeth-Ayerst Company) as a tetravalent attenuated vaccine containing G serotypes(G1 to G4) that were the most commonly found in the world was approved by US FDA in 1998 and contained in basic inoculations that are applied to babies who become 2, 4 and 6 months but its use was stopped because 15 cases of intussusceptions were happened (Murphy TV et al., N Engl J Med 344:564-572, 2001).

Therefore, due to problems mentioned above, there has been a need for the development of an effective vaccine strain against human rotavirus and it is necessary to study on gene analysis and diagnosis of human rotavirus prior to the development. The present inventors have detected human rotavirus for diagnosis of human rotavirus and development of human rotavirus vaccine, have identified that the detected human rotavirus is a combined human rotavirus having a G11P[4] serotype by analyzing VP7 and VP4 genes as coat genes of human rotavirus, and have completed the invention by confirming that the virus is a novel human rotavirus that was not reported previously through phylogenetic analysis of VP7 gene.

Thus, it is an object of the present invention to provide a VP7 gene encoding the amino acid sequence set forth in SEQ ID NO:2.

It is another object of the present invention to provide a composition for diagnosis of human rotavirus infection comprising primer or probe specific to the VP7 gene.

### BRIEF DESCRIPTION OF THE DRAWINGS

FIG. 1 shows results of phylogenetic analysis for VP7 nucleotide sequence of human rotavirus CUK-1 detected in the present invention with 15 kinds of G type rotaviruses.

FIG. 2 shows results of comparison of amino acid sequences for four intragenotype-conserved antigenic regions in human rotavirus CUK-1 detected in the present invention and 15 kinds of G type rotaviruses.

### DETAILED DESCRIPTION OF THE INVENTION

In order to achieve the above objects of the invention, the present invention provides a VP7 gene encoding the amino acid sequence set forth in SEQ ID NO:2.

The present invention also provides a composition for diagnosis of human rotavirus infection comprising a primer or a probe specific to thereof.

The present invention will be described in detail.

The present invention provides a VP7 gene encoding the amino acid sequence set forth in SEQ ID NO:2.

The genes forming the coat of rotavirus are VP7 and VP4 genes, and related to pathogenicity, immunogenicity, and cell adhesion and infestation of viruses. Rotavirus G serotypes are classified by the VP7 gene and rotavirus P genotypes are classified by the VP4 gene.

In embodiments of the present invention, rotavirus is detected from a feces sample of patients showing rotavirus infection symptoms using a VP6 specific ELISA kit, and VP7 and VP4 genes are analyzed from the detected sample through a series of processes including virus RNA extraction, RT-PCR and nucleotide sequence analysis. As a result, human rotavirus of the present invention is identified as a novel G11 P[4] type human rotavirus and is referred to as “CUK-1”. Meanwhile, the nucleotide sequence of VP7 gene of human rotavirus CUK-1 analyzed above is represented by SEQ ID NO:1 and the nucleotide sequence of VP4 gene is represented by SEQ ID NO:3. Additionally, the amino acid sequence of VP7 protein encoded by the VP7 gene is represented by SEQ ID NO:2, and the amino acid sequence of VP4 protein encoded by the VP4 gene is represented by SEQ ID NO:4.

The present invention also provides a composition for diagnosis of human rotavirus infection comprising primer or probe specific to thereof.

The detection of a specific nucleic acid using primer can be performed by amplifying the nucleotide sequence of a target gene using an amplification method such as PCR and confirming the amplification of the target gene according to method previously known in the art. Additionally, the detection of a specific nucleic acid using probe can be performed by contacting a nucleic acid sample with the probe under an appropriate condition and confirming the presence of hybridized nucleic acid.

The term of “primer” as used herein means a short nucleotide sequence having a short free hydroxide group, forming a base pair with a complementary template, and functioning as an origin for replication of a template strand. The primer of the present invention, for example, can be synthesized chemically according to method previously known in the art such as phosphoramidite solid support method.

The term of “probe” as used herein means a nucleic acid fragment such as RNA or DNA consisting of numerous bases capable of specifically binding to mRNA, and is labeled to confirm the presence of specific mRNA. The probe can be manufactured in a type of oligonucleitide probe, single chain DNA probe, double chain DNA probe or RNA probe, and be labeled with biotin, FITC, rhodamine, DIG or radioactive isotope.

Additionally, the probe can be labeled with a detectable material, for example, a radioactive marker providing an appropriate signal and having a sufficient half-life. The labeled probe can be hybridized with a nucleic acid on a solid support as published in a reference (Sambook et al., Molecular Cloning, A Laboratory Mannual, 1989).

Examples of methods for detecting a specific nucleic acid using the probe or primer include, but are not limited to, polymerase chain reaction (PCR), DNA sequencing, RT-PCR, primer extension method (Nikiforeov et al., Nucl Acids Res 22, 4167-4175, 1994), oligonucleotide extension analysis (Nickerson et al., Pro Nat Acad Sci USA, 87, 8923-8927, 1990), allele specific PCR (Rust et al., Nucl Acids Res, 6, 3623-3629, 1993), RNase mismatch cleavage (Myers et al., Science, 230, 1242-1246, 1985), single strand conformation polymorphism (Orita et al., Pro Nat Acad Sci USA, 86, 2766-2770, 1989), denaturing gradient gel electrophoresis (DGGE) (Cariello et al., Am J Hum Genet; 42, 726-734, 1988), denaturing high performance liquid chromatography (Underhill et al., Genome Res, 7, 996-1005, 1997), hybridization, DNA chip, etc. Examples of the hybridization include Northern hybridization (Maniatis T. et al., Molecular Cloning, Cold Spring Habor Laboratory, NY, 1982), In situ hybridization (Jacquemier et al., Bull Cancer, 90:31-8, 2003), microarray (Macgregor, Expert Rev Mol Diagn 3:185-200, 2003), etc.

The composition for diagnosis of human rotavirus infection further comprises reagents commonly used in the specific nucleic acid-detecting methods mentioned above. For example, the composition further comprises dNTP(deoxynucleotide triphosphate), heat resistant polymerase and metal ion salts including magnesium chloride which are required for PCR, or dNTP and sequenase which are required for sequencing.

Preferably, the diagnostic composition of the present invention is provided in types of diagnostic kit, microarray and DNA chip.

### BEST MODE FOR CARRYING OUT THE INVENTION

Hereinafter, the present invention will be described in further detail by the following examples. It is to be understood, however, that these examples are given for illustrative purpose only and are not intended to limit the scope of the present invention.

### EXAMPLE 1

### Detection of Human Rotavirus

A sample was collected from feces of child patients becoming less than 5 years old suffering from diarrhea in our lady of mercy hospital in Incheon, Korea. The rotavirus antigen was detected from the sample using a VP6-specific ELISA (Enzyme-linked immunosorbant assay) kit, Dipstick ‘Eiken’ Rota kit (SA Scientific, Eiken, Tokyo, Japan). The detected human rotavirus was referred to as CUK-1.

### EXAMPLE 2

### Analysis of Nucleotide Sequence of Human Rotavirus VP4 and VP7 genes

### <2-1> RNA Extraction

The virus RNA was extracted from the feces sample in which the rotavirus antigen was detected using QIAamp® virus RNA mini-kit (QIAGEN/Westburg, Leusden, The Netherlands). First, the feces sample was suspended in PBS (phosphate-buffered saline) to prepare 10%(w/v) suspension, and centrifuged at 3000×g for 20 minutes. After centrifugation, the supernatant was collected and virus RNA was extracted from the supernatant using QIAamp® virus RNA mini-kit. The extracted RNA was stored at −70° C.

### <2-2>RT-PCR

In order to identify G genotype and P genotype of rotavirus, RT-PCR (Reverse transcriptase PCR) was carried out using a one step RT-PCR kit.

In order to amplify the VP7 gene, RT-PCR was carried out using a sense primer Beg9 (5′-GGCTTTAAAAGAGAGAATTTCCGTCTGG-3′: SEQ ID NO:5) and an antisense primer End9 (5′-GGTCACATCATACAATTCTAATCTAAG-3′: SEQ ID NO:6). The reaction was incubated at 50° C. for 30 minutes as an initial reverse transcription step, followed by PCR activation at 95° C. for 15 minutes, 35 cycles of amplification at 94° C. for 45 seconds, 54° C. for 50 seconds and 72° C. for 45 seconds, and incubation at 72° C. for 10 minutes to amplify the VP7 gene consisting of 1,062 bp. The amplified PCR product was then electroporated on agarose gel, stained with EtBr (ethidium bromide) and confirmed on UV.

In order to amplify the VP4 gene, RT-PCR was carried out using a sense primer Con3 (5′-TGGCTTCGCCATTTTATAGACA-3′: SEQ ID NO:7) and an antisense primer Con2 (5′-ATTTCGGACCATTTATAACC-3′: SEQ ID NO:8). The RT-PCR reaction was incubated at 50° C. for 30 minutes as an initial reverse transcription step, followed by PCR activation at 95° C. for 15 minutes, 35 cycles of amplification at 94° C. for 45 seconds, 50° C. for 50 seconds and 72° C. for 45 seconds, and incubation at 72° C. for 10 minutes to amplify the VP4 gene consisting of 876 bp. The amplified PCR product was confirmed by performing electrophoresis on 1.5% agarose gel.

### <2-3> Analysis of Nucleotide Sequence

In order to analyze the nucleotide sequences of VP7 and VP4 genes, the amplified PCR product in Example <2-2> was purified with the QIAEX II gel extraction kit (QIAGEN, Hilden, Germany). 60 ng of the purified PCR product, 5 pmol of sense or antisense primer and 4 μl of Big Dye Terminator v2.0 100RR mix (Perkin-Elmer Applied Biosystems) were mixed, and final volume of the mixture was regulated to 10 μl with sterilized water, followed by 30 cycles of 96° C. for 10 seconds and 60° C. for 5 seconds, followed by 60° C. for 4 minutes. The nucleotide sequences of both strands are analyzed and cross-checked.

The analyzed VP7 gene nucleotide sequence is represented by SEQ ID NO:1 and VP4 gene nucleotide sequence is represented by SEQ ID NO:3, and VP7 amino acid sequence and VP4 amino acid sequence encoded by the nucleotide sequences are represented by SEQ ID NO:2 and SEQ ID NO:4, respectively.

### EXAMPLE 3

### P Type Determination of Human Rotavirus VP4 Gene

In order to determine P type of human rotavirus VP4 gene, the nucleotide sequence of the VP4 gene determined in Example 2 was compared with related strains in GenBank, and the human rotavirus CUK-1 according to the present invention was identified as P[4] type human rotavirus.

### EXAMPLE 4

### G Type Determination and Genetic Correlation Analysis of Human Rotavirus VP7 Gene

In order to determine G type and analyze genetic correlation of human rotavirus VP7 gene, the nucleotide sequence of the VP7 gene determined in Example 2 was compared with the related strains in GenBank. DNA and protein sequences were aligned using multiple alignment algorithm of MegAlign package (Window version 3.12e; DNA STAR, mADISON, Wis.). Additionally, homology and sequence distance between nucleotide and amino acid sequences were calculated by Cluster W method, and phylogenetic analysis was carried out using Mega version 3.1 software package. The DNA sequence was calculated by Cluster W method, and dendrogram was calculated by neighbor-joining method.

TABLE 1
Comparison of homology of nucleotide and amino acid sequences between
VP7 gene of 15 kinds of G genotype rotaviruses and VP7 gene of present
CUK-1 rotavirus strain

| | | | Homology (%) | |
|---|---|---|---|---|
| | | | Nucleotide | Amino Acid |
| G genotype | Strain | Origin | Sequence | Sequence |
| G1 | Wa | Human | 73.8 | 77.6 |
| G2 | HU5 | Human | 74.3 | 74.2 |
| G3 | SA11 | Monkey | 76.8 | 86.2 |
| G4 | ST3 | Human | 73.8 | 76.1 |
| G5 | OSU | Pig | 83.6 | 89.9 |
| G6 | NCDV | Cow | 74.4 | 81.3 |
| G7 | Ch2 | Birds | 62.1 | 57.7 |
| G8 | MW333 | Human | 73.4 | 80.1 |
| G9 | 116E | Human | 78.9 | 84.7 |
| G10 | B223 | Cow | 72.5 | 77.9 |
| G11 | A253 | Pig | 86.9 | 95.1 |
| G11 | Dhaka6 | Human | 98.7 | 98.5 |
| G11 | YM | Pig | 91.9 | 95.4 |
| G12 | L26 | Human | 74.6 | 80.4 |
| G13 | L338 | Horse | 74.4 | 78.5 |
| G14 | F123 | Horse | 76.9 | 82.2 |
| G15 | Hg18 | Cow | 73.4 | 77.3 |

(ORF (open reading frame) of VP7 consisting of 49th to 1026th nucleotides was sequenced.)

The nucleotide sequence of VP7 gene ORF (open reading frame) of the present invention was compared with that of G1 to G15 types VP7 gene ORF. As shown in Table 1 described above, the VP7 nucleotide sequence of the present CUK-1 strain was the most closely related to G11 type rotavirus strains including A253, Dhaka6 and YM (Homologies of nucleotide sequence were 86.9%, 98.7% and 91.9%, respectively, and homologies of amino acid sequence were 95.1%, 98.5% and 95.4%, respectively). Thus, the VP7 nucleotide sequence of the present CUK-1 strain showed the highest homology to Dhaka6 strain, and low homologies to G1 to G15 types except G11 type. Based on these results, CUK-1 strain of the present invention was identified as G11 type human rotavirus.

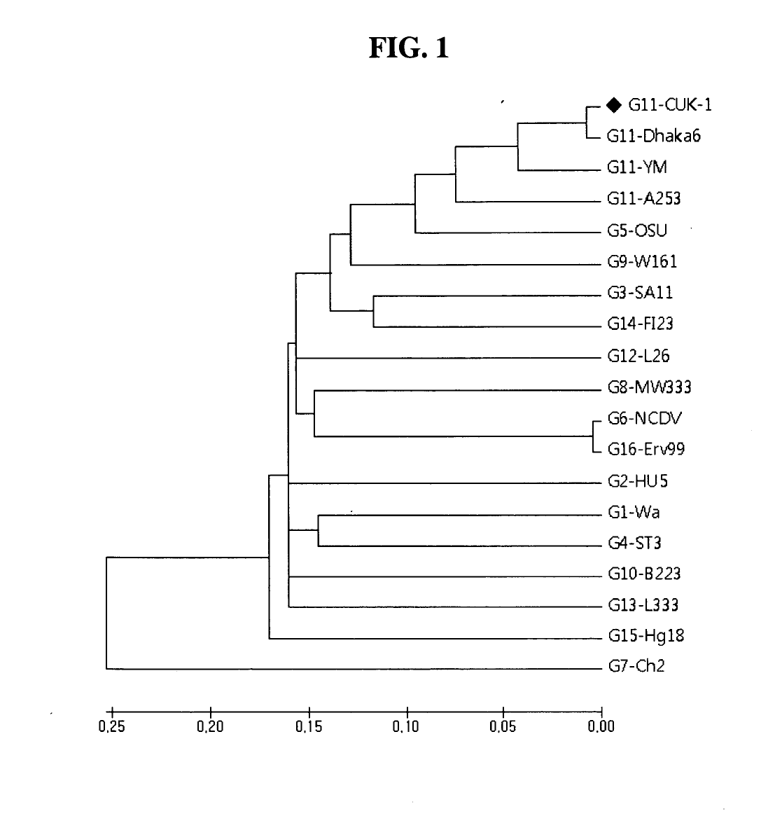

Meanwhile, as shown in FIG. 1 showing results of phylogenetic analysis for VP7 nucleotide sequence of human rotavirus CUK-1 with 15 kinds of G type rotaviruses, CUK-1 and G11 type Dhaka6 strain were clustered within a unit branch.

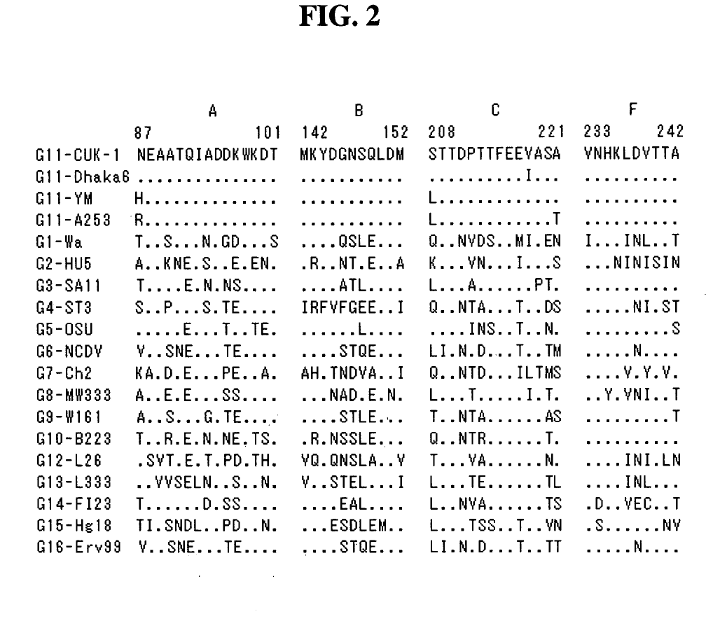

Additionally, the amino acid sequences of four intragenotype-conserved antigenic regions in human rotavirus CUK-1 and 15 kinds of G type rotaviruses were aligned. As a result, as shown in FIG. 2, CUK-1 strain showed a close relation to G11 type rotavirus strains including A253, Dhaka6 and YM. Comparing with Dhaka6, one amino acid sequence in region “C” was different.

### EFFECTS OF INVENTION

As discussed above, the present invention detects human rotavirus having combinations of G11P[4] types. As a result of VP7 gene analysis, it is confirmed that the virus is a novel human rotavirus that was not reported previously. Thus, the human rotavirus VP7 gene according to the present invention will be useful for diagnosis of novel G11 type human rotavirus infection, and will be used for development of rotavirus vaccine.

What is claimed is:

1. A VP7 gene of human rotavirus encoding the amino acid sequence set forth in SEQ ID NO:2.

2. The gene according to claim 1, wherein the gene has the nucleotide sequence set forth in SEQ ID NO:1.

3. A VP7 polypeptide of human rotavirus having the amino acid sequence set lo forth in SEQ ID NO:2.

4. A composition for diagnosis of human rotavirus infection comprising a primer or a probe specific to the gene according to claim 1.
